(19) United States
(12) Patent Application Publication
(10) Pub. No.: US 2005/0266069 A1
(43) Pub. Date: Dec. 1, 2005

(54) Stable probiotic microsphere compositions and their methods of preparation

(76) Inventors: Donald L. Simmons, Dollard des Ormeaux (CA); Peyman Moslemy, Cote St-Luc (CA); Gilles D. Paquette, Laval (CA); Daniel Guerin, Laval (CA); Marie-Helene Joly, Laval (CA)

Correspondence Address: MYERS BIGEL SIBLEY & SAJOVEC, PO BOX 37428, RALEIGH, NC, 27627, US

(21) Appl. No.: 10/656,386

(22) Filed: Sep. 5, 2003

Related U.S. Application Data
(60) Provisional application No. 60/408,348, filed on Sep. 6, 2002.

(51) Int. Cl. A61K 45/00; A61K 9/48

(52) U.S. Cl. 424/451; 424/93.45

(57) ABSTRACT

The invention relates to viable and stable probiotic formulations for intestinal targeting made of microspheres comprising each a core of one or more probiotic bacteria, microcrystallline cellulose with a degree of polymerization from 165-365 and mean diameter from 45 to 180 μm, a disintegrant and a stabilizer, the core being coated with a non-enteric coating and further coated with an enteric coating. Each probiotic microsphere has a residual moisture level of less than 5% and a water activity (a_{w}) between 0.1 and 0.5. Such a probiotic microsphere shows no reduction in viable bacteria after one hour in simulated gastric fluid. The present invention also relates to the process of preparing such formulation.

### RELATED APPLICATIONS

This application claims priority from U.S. Provisional Patent Application No. 60/408,348, filed on Sep. 6, 2002, the disclosure of which is incorporated by reference herein in its entirety.

### BACKGROUND OF THE INVENTION

A) Field of the Invention

This invention relates to viable probiotic microsphere core compositions, and methods for making thereof. The invention further relates to stable coated probiotic microsphere compositions for targeting to specific regions of the intestinal tract.

B) Brief Description of the Prior Art

Efficacy Considerations with Probiotics

Probiotics are defined as live microbial dietary adjuvants that beneficially affect the host physiology by modulating mucosal and systemic immunity, as well as improving intestinal function and microbial balance in the intestinal tract (Naidu et al. Critical Reviews in Food Science and Nutrition, 1999, 38(1): 13-126). In order to exert their beneficial effects on the host, probiotics must remain viable and reach the large intestine in large numbers (Favaro-Trindade C. S. et al, 2002, J Microencapsulation 19(4): 485-494)

Lactic acid bacteria (LAB) as probiotics for human consumption are most frequently incorporated into dairy products such as yogurts, fermented milks and kefirs. However, their use is becoming ever more widespread for example in dietary supplement form as powders, capsules and tablets. Lactobacilli remain the most commonly used probiotic microorganisms followed closely by Bifidobacteria and Streptococci. Various nutritional and therapeutic effects have been ascribed to these probiotics including: modulating immune response, lowering serum cholesterol concentrations, improving lactose intolerance symptoms, increasing resistance to infectious intestinal diseases, decreasing diarrhoea duration, reducing blood pressure, and helping to prevent colon cancer.

Despite this impressive list of prophylactic and therapeutic attributes, probiotics remained until recently a conundrum to the scientific and medical communities. The current scientific literature is now replete with animal data that successfully demonstrates the anticarcinogenic and antimutagenic potential of probiotics, especially Bifidobacterium longum, alone or in combination with prebiotics, i.e., synbiotics. Prebiotics are defined as non-digestible carbohydrates, e.g., oligosaccharides, which pass through the small intestine undigested and serve as selective nutrients for friendly bacteria already resident in the colon. Rowland et al (Carcinogenesis, 1998, 19(2): 281-285) investigated the effect of administering B. longum and inulin, alone or in combination, to the diet of rats injected with the carcinogen azoxymethane (AOM) to induce colonic aberrant crypt foci (ACF). The latter are preneoplastic lesions from which carcinomas may develop in the colon. Decrease in ACF numbers of 26% and 41% versus controls were obtained following consumption of B. longum and inulin, respectively. However an 80% inhibition of similar small ACF was obtained following consumption of the combination. Of prime interest was the observation that the combination significantly decreased the incidence (by 59%) of large ACF (>4 aberrant crypts per focus), which are considered to be predictive of eventual tumour incidence.

In a similar experiment involving ACF incidence, Gallaher and Khil (Journal of Nutrition, 1999, 129(7 Supplement): 1483S-1487S) administered bifidobacteria and oligofructose, alone or in combination, along with the carcinogen 1,2-dimethylhydrazine (DMH) to the diets of rats. Significant reduction in ACF incidence was not observed in the bifidobacteria and oligofructose treatments alone. However, a paired comparison of the six conducted experiments indicated a significant overall reduction in ACF by the combination. Similar results were obtained in animal investigations involving B. longum and other prebiotics, for example: short-chain fructooligosaccharides (Koo & Rao, Nutrition and Cancer, 1991, 16(3-4): 249-267); lactulose (Challa et al, Carcinogenesis, 1997, 18(3): 517-521).

Reddy et al (International Journal of Oncology, 1999, 14(5): 939-944) also demonstrated in rats that a specific strain of L. acidophilus NCFMTM significantly suppressed AOM-induction of colonic ACF. McIntosh et al (Nutrition and Cancer, 1999, 35(2): 153-159) investigated several probiotic strains for their influence on 1,2-DMH-induced intestinal tumors in 100 Sprague-Dawley rats. The large intestinal tumor burden was significantly lower for treated rats with Lactobacillus acidophilus versus controls but not for Lactobacillus rhamnosus, Bifidobacterium animalis or Streptococcus thermophilis.

Utilizing a standard Ames test, Lankaputhra and Shah (Mutation Research, 1998, 397: 169-182) determined the antimutagenic activities of live and killed cells of 6 strains of L. acidophilus and 9 strains of bifidobacteria using 8 potent chemical mutagens and promutagens. The authors found that live cells of all strains studied showed higher levels of inhibition than the killed cells. Recovery of mutagens from live cells was less than 5% in contrast to 80-95% recovery from killed cells. The results emphasized the importance of consuming live probiotic bacteria and of maintaining their viability in the intestine in order to provide efficient inhibition of mutagens. Therefore careful attention to probiotic strain(s) selection and maintaining their viability in products to the consumer is an important issue to be addressed by scientific and clinical researchers involved in the probiotic industry.

Stability Considerations with Probiotics

The viable microbial content and general quality of many probiotic-containing products have long been problematic, and evidence for this dilemma exists in the scientific literature. In one study reporting on L. casei content in commercial yogurts, Shah (Journal of Dairy Science, 2000, 83(4): 894-907), found no traces of live microorganisms in 3 of 6 products tested and only low concentrations in two others. Similar discrepancies have been reported with commercial probiotic products distributed in solid dosage forms such as powders, capsules and tablets. Hughes et al (Obstetrics Gynecology, 1990, 75: 244-248) analyzed 11 products, claiming to contain L. acidophilus, for number and type of bacteria present. Only two products contained L. acidophilus while the remainder contained L. casei.

Similar problems in maintaining L. acidophilus viability contained in gelatin capsules are also evident from the patent literature. For example Langner in U.S. Pat. No. 6,008,027 describes gelatin capsules filled with 90% freeze-dried L. acidophilus culture and 10% silicon dioxide, enteric coated with polyvinyl acetate phthalate (PVAP) and then vacuum dried for 72 hours to remove moisture and oxygen. After only one-year storage at room temperature, the bacterial colony counts were reduced from an initial concentration of 1 billion colonies per gram to only 33 million colonies per gram. Kim et al (U.S. Pat. No. 6,365,148) disclosed enteric-coated microgranules for stabilizing lactic acid bacteria. Utilizing a fluidized bed granulator, water soluble and enteric polymers were applied directly to probiotic bacteria alone or admixed with mostly water-soluble additives. Such core granules are expected to be extremely hard and undergo slow disintegration.

Fusca et al. (U.S. Pat. No. 6,254,886) describe a large multilayer tablet containing probiotics in one layer and foodstuff ingredients in other layers. The moisture content of added excipients for the probiotic layer was established below 0.1% with the assurance that, in the absence of special cooling conditions, the tablets can be stored in their “packs” on pharmacists shelves for a period of 6 months to a year without loss of activity. The tablets disclosed by Fusca et al. are large (approximately 1 gram weight) having no barrier and enteric coat protection.

Probiotics are also commercially available in seamless or soft gelatin capsules. Bifa-15™ (Eden Foods, Clinton, Mich.,) contains 1 billion viable Bifidobacterium longum dispersed in coconut oil within small enteric-coated seamless capsules. The capsules are admixed with oligosaccharides, sweeteners and flavours and presented in individually wrapped, single dose aluminum tubes. The contents are poured into the mouth with the proviso that capsules be swallowed whole and not chewed. Ultra-Dophilus™ (Nature's Plus, Melville, N.Y.,) is a conventional-sized soft gelatin capsule containing 2 billion viable L. acidophilus and 100 mg of apple pectin dispersed in safflower oil. Although these products bear labels with statements to the effect that declared viability is at the time of manufacture, no guarantee exists that the label claim will be met following storage at room temperature, e.g., 22-25° C. at some future time period.

Therefore providing bacterial viability and stability upon prolonged storage is an important need that has yet to be met and continues to challenge the industry. Researchers now have an important testing tool to expedite the generation of stability data. This program, dubbed ASLT or Accelerated Shelf Life Testing, was initiated and championed by Professor Ed Garrett at the University of Florida in Gainesville more than 40 years ago. His thermal degradation investigations using the Arrhenius relationship dealt mainly with drugs or vitamins but the technology has now been adapted for evaluating probiotic formulations. Mitic et al (Cryobiology, 1974, 11: 116-120) predicted the stabilities of freeze-dried suspensions of L. acidophilus by using this thermal degradation-testing program. Bacterial suspensions in skim milk were distributed in ampoules, freeze-dried, vacuum-dried and the ampoules sealed under vacuum. The ampoules were then submerged in thermostatically controlled water baths maintained at 50, 60 and 70° C. At predetermined time intervals, samples were removed and analyzed for bacteria viability. Thermal bacteria death rates were obtained from the negative slopes resulting from linear plots of log₁₀ number of organisms remaining versus time. The logarithms of the resulting rate constants are then plotted against the reciprocal of the absolute temperatures. Extrapolation along this straight line allowed the authors to predict the log loss values (expressed as colony-forming-units per gram or cfu/g) of the samples laid aside at 4 and 20° C. at various monthly intervals. For example after 19 months at 20° C, the predicted and actual found log loss values for remaining organisms were 1.25 and 1.30, respectively. More recently Kim et al (Journal of General Applied Microbiology, 1998, 44: 161-165) utilized the same methodology to predict shelf lives of both freeze-dried and controlled low-temperature vacuum dehydrated L. acidophilus samples.

Encapsulation Considerations with Probiotics

Spheres, as oral dosage forms, offer numerous advantages over more conventional single-unit systems such as tablets and capsules. Their small size, round shape and divided nature permit greater ease in controlling release patterns of bioactive agents, more uniform coating applications, and consequently superior potential for intestinal targeting. Although various processes are used to produce spheres, arguably the best results appear to be achieved with extrusion followed by spheronization. This process comprises a series of non-continuous stages known as “granulation”, “extrusion”, “spheronization” and “drying”. The process begins with a granulation step whereby a bioactive agent, stabilizers and other ingredients are mixed with a liquid binder, usually water, to form a wet powder mass or paste. The next step involves screw or ram extrusion of the wet mass through a die to form cylindrical strands of uniform length and diameter. These strands are then made into spheres by first chopping them into equal lengths before rounding into pellets of spherical shape on a marumerizer plate. Alternatively, the wet mass may be extruded through the screen of an oscillating granulator and the resulting jagged cylinders formed into spheres by rotation in a conventional coating pan under forced-air drying conditions. The final step involves collecting the wet spheres and drying them in either a fluidized bed or tray drier prior to use.

Successful extrusion-spheronization must satisfy a number of requirements. Firstly, it requires the production of a cohesive wet mass that is able to flow through a die without adhering to the extruding device but with a sufficient degree of rigidity so that the shape imposed by the die is retained. An additional requirement is that the extrudate be brittle enough to break into uniform lengths onto the spheronization plate but still be plastic enough to round into spherical pellets. However, most bioactive agents do not possess such ideal characteristics. Addition of certain excipients, such as microcrystalline cellulose (MCC), is therefore needed to produce formulations with the necessary rigidity-plasticity and water-absorbing capacity required for successful extrusion-spheronization.

The unique functionality of MCC in the extrusion-spheronization process is still not fully understood. Water, which is known to play a major role in the process, is typically used as a granulation fluid. However the quantity and distribution of water in the wet mass during the extrusion phase is crucial to the success of the spheronization process that follows. An additional factor in achieving a successful end product using extrusion-spheronization technology is the type and the physicochemical properties of the cellulose materials used in the process.

In one report (Kleinebudde et al. MPS PharmSci, 2000, 2(2) article 21: 1-10), the extrusion process was evaluated using powdered cellulose and two commercial MCC materials that differed in degrees of polymerization (DP values). The MCC materials possessed DP values of 225 and 166 (Avicel™ PH 101 and Avicel™ PH 301 respectively; FMC Corporation, Philadelphia, Pa.) and a DP value of 1431 for the powdered cellulose (Elcema™ P050; Degussa, Frankfurt am Main, Germany). The materials were to be extruded at a desired power consumption of 200 W in a twin-screw extruder, spheronized and dried. The denser material with DP 166 was easily extrudable but yielded big agglomerates and needed to be extruded at 250 W to provide pellets of questionable quality. The extrusion of DP 225 proceeded without incident during extrusion at 200 W and yielded round pellets. The extrusion of powdered cellulose with DP 1431 was beset with difficulties such as operating at maximum frequency, obstruction of the dies, powder accumulation in the barrel, etc.

To date, extrusion-spheronization technology has been almost exclusively associated with drugs and efforts to improve their clinical performance through controlled or delayed release action. However Sipos (U.S. Pat. No. 4,079,125) utilized the extrusion-spheronization technology, combined with enteric coating, for enzyme delivery but the requirement of isopropyl alcohol as solvent in the processing operation led to a wide range of particle sizes and such solvents are not suitable for processing probiotics. Brown et al. (U.S. Pat. No. 6,060,050) applied a modified mixing and extrusion technology to entrap probiotics in high amylose starches, such as wheat, corn and barley, wherein probiotic plus a low-melting fat and starch plus low-melting fat mixtures are separately fed into a screw type extruder at a temperature slightly above the fat melting point. The resulting suspension is then successively forced through a chilled die fitted with 2-mm diameter orifices, granulated and further refrigerated in order to maintain granule shape until use. These granules however proved to have only limited shelf life upon storage at ambient temperatures.

More recently, van Lengerich (U.S. Pat. No.6,500,463) disclosed a similar mixing and extrusion technology to entrap L. acidophilus. A homogeneous dry blend—consisting of gluten-containing durum semolina, non-gelatinized wheat starch, low melting fat and L. acidophilus powder—along with water are separately fed and extruded through a twin screw extruder at a temperature just below fat melting temperature. The extruded strands are cut into pellets approximately 0.5 to 1.5 mm in length and then air dried for about 30 minutes to obtain shelf-life stable pellets containing stable and protected active L. acidophilus. No evidence exists for such stability claims and the presence of gluten-containing materials is detrimental to the health of people who suffer from celiac disease.

Kim et al. (Journal of Industrial Microbiology, 1988, 3: 253-257) applied the conventional extrusion-spheronization process to encapsulate Lactobacillus plantarum. The procedure consists of mixing 50 parts dry culture microorganism containing 2-4% of non-fat milk solids with 50 parts powdered cellulose. The mixture is granulated with one part glycerol in 54 parts distilled water to a final wet granulation containing 37% water. The wet mass is then extruded through a screen (1.2 mm orifices) and the extruded filaments spheronized on a rotating plate. The resulting spheres are dried in a fluidized bed dryer, screened and coated with various polymers. The screening and collection of spheres in the 300 to 1,700 μm in diameter range for coating applications indicated a wide size distribution profile for spheres prepared using powdered cellulose as carrier. The extrusion,: spheronization and drying methods were not detrimental to the viability of the cultures. However, the application of sodium alginate and carboxymethylcellulose coatings to these spheres offers little protection to cell viability. Greater than 4 log reduction for both uncoated and coated spheres was observed after only 14 days at 37° C. The relatively high residual moisture content (10%) may explain, in part, this poor viability performance.

Also known in the prior art relating to the field of food industry are studies on the importance of water activity on the viability of bacteria. Water activity measures the vapour pressure generated by the moisture present in a hygroscopic product i.e. a product capable of readily absorbing moisture from the air. Water activity reflects the part of moisture content of a product which can be exchanged between the product and its environment. It represents the ratio of the water vapour pressure of the product to the water vapour pressure of pure water under the same conditions. It is expressed as a fraction a_{w}=p/ps

Where

p: is the partial pressure of water vapour at the surface of the product
ps: is the saturation pressure of the partial pressure of water vapour above pure water at the product temperature.

Water activity has found useful application in the food industry particularly in predicting the growth of bacteria. Controlling the water activity can effectively control the product's stability and can make it possible to predict the shelflife under known ambient storage conditions.

A number of electronic instruments exist for rapidly and precisely measuring a_{w} of products.

Champagne et al. (Bioscience Microflora, 1996, 15(1): 9-15) recognized the importance of a_{w} on lactic acid bacteria (LAB) stability in a study involving the a_{w} effect of various polymers on bacteria survival during storage. Operating on a literature claim that stability of LAB (including B. longum) upon storage is best in the 0.1 to 0.22 range, the investigators stabilized all samples at 0.22 a_{w} or 23% relative humidity using a saturated potassium acetate solution. In a related study, Castro et al (Applied Microbiology and Biotechnology, 1995, 44: 172-176) investigated the viability of lyophilized cultures of Lactobacillus bulgaricus in skim milk during storage at different temperatures, relative humidities and atmospheres. Best survival results occurred with samples stored at 11 and 23% relative humidity, under nitrogen and at the lowest temperatures. It was concluded that the oxidation of lipid components of the cell membrane is accelerated at higher a_{w} levels obtained in higher relative humidities, leading to accelerated death of bacteria. Laroche and Gervais (Applied and Environmental Microbiology, 2003. 65(5): 3015-3019) reported on the thermal destruction of dried, glass bead-immobilized L. plantarum samples with water activities ranging between 0.10 and 0.70. The authors concluded that the viability of L. plantarum is closely dependent on a_{w}, and the microorganism shows an optimum resistance to heat stress at an a_{w} around 0.35.

Intestinal Targeting Considerations with Probiotics

The rationale behind specific organ delivery of orally administered drugs evolved almost 30 years ago when it became clear that some laxative drugs are active only after arrival at the large intestine. This observation led to research activity into the development of colonic dosage forms utilizing two major approaches: (a) protective coatings to bring the dosage form as close as possible to the colon after oral administration, e.g., time-dependent delivery systems, and (b) use of prodrugs, polymeric prodrugs and special polymers that are degraded mostly by colon enzymes, e.g., azopolymer coatings degraded by the colon enzyme azoreductase. However, extensive toxicity testing is required on such polymers before their acceptance by the regulatory agencies.

Time-dependent delivery systems rely on complete and rapid bioactive release following a well-defined lag-time without release. These delivery systems consist of dosage form units coated with a barrier layer that slowly dissolves or erodes after a specified lag period. Lag times are controlled by the thickness of the barrier layer. Following complete erosion of the barrier layer, the contents are rapidly released from the dosage form. Poli et al (EP Patent 1993, 0,572,942) utilized this delivery system to target various drugs with known indications for treating diseased colon conditions. Such systems are ideally suited for bioactives such as probiotics, which require a sufficient lag time to transit the hostile environments of the stomach and upper intestinal tract, followed by targeting to specific sites in the small and large intestines.

Passage time of bioactive dosage forms from mouth to colon is now well understood. Although gastric emptying is highly variable, small intestine transit time is remarkably constant at 3 to 4 hours. Colon transit times are highly variable and influenced by numerous factors such as diet, stress, drugs, mobility and disease. In an updated review on colonic drug delivery, Shareef et al (MPS PharmSci, 2003, 5(2) Article 17 [http://www.pharmsci.org], 161-186) reported that larger dosage forms had faster transit times than their smaller counterparts. The authors postulated that the retention time of bioactive dosage forms within the colon is better achieved with multiparticulate units such as spheres rather than with a single unit such as tablet or capsule. This delay with spheres is additional assurance that the units do not pass too quickly through the colon before releasing total contents.

Orally ingested probiotics, like enzymes and some drugs, undergo extensive degradation on entry into the stomach because of exposure to acid, pepsin and other gastric enzymes. Further attack on their integrity occurs after passage into the duodenum where they are exposed to bile from the liver and gallbladder, as well as pancreatic juices from the pancreas. A minimum requirement therefore for any oral probiotic dosage form is enteric coating.

Many manufacturers produce and distribute probiotic products that do not possess gastric protection. Their rationale is to include an excessive quantity of probiotics in the product in anticipation that a major portion will survive and reach their target. In addition to questionable shelf-life viability for these products, such practices are not cost-effective. Some manufacturers do enteric-coat their products, for example, Jarro-Dophilus EPS (Jarrow Formulas, Los Angeles, Calif.) contains a total of 4.4 billion organisms per capsule comprising 4 different strains of lactobacilli, 2 strains of bifidobacteria plus Pediococcus acidilactici and Lactococcus diacetylactis. Specific organ targeting of these different organisms is not possible since they will all have a common release site following erosion of the applied film and capsule shell. The net result is dilution and competition among the various strains at the release site.

A major impediment in developing target-specific dosage forms is the lack of an adequate in vitro testing system capable of differentiating between formulation variables. One testing system of interest is an assembly used successfully in designing, formulating and screening conventional and sustained-release oral dosage forms (Simmons et al, Canadian Journal of Pharmaceutical Sciences, 1980, 15(2), 26-29). Dissolution rate data from the assembly was capable of predicting serum or plasma drug concentration time profiles in humans. The major feature of the assembly is that it was designed to include two distinct compartments operating in unison. The first compartment, or disintegration chamber, provides turbulent hydrodynamic conditions around the dosage form approximating the conditions known to exist in the stomach. Following disintegration, the fragmented granules or particles are expelled through small apertures (e.g., pyloric sphincter) of the disintegration chamber into an aqueous environment providing mild uninterrupted hydrodynamic conditions as found in the upper intestinal tract. Therefore adapting an in vitro release system for probiotic products to approximate the corresponding release conditions in the gastrointestinal tract is an additional burden on scientists working in the probiotic industry.

In view of the above, the prior art does not provide a common solution to the separate problems of bacterial cell viability, bacterial cell stability, particle size uniformity, residual moisture content, water activity a_{w}, intestinal targeting and limited shelf life facing the probiotic product industry.

Thus there exists a definitive need for viable probiotic microsphere compositions comprising one or more probiotic bacteria.

There is a need also for processes that are cost-effective and capable of entrapping and stabilizing probiotics in microspheres with minimal viability loss at the end of the entire operation.

There is also a need for processes for producing uniform microsphere compositions within a narrow size distribution range.

A need also exists for processes capable of producing microsphere compositions with low residual moisture contents and water activity (a_{w}) values.

There is a definite need also for coated microsphere compositions containing probiotics targeted to specific regions of the intestinal tract.

As can also be seen from the foregoing, there is a long felt need for oral probiotic compositions that are stable at room temperature for greater than 18 months.

The present invention fulfills these needs and also other needs, which will be apparent to those skilled in the art upon reading the following specification.

### SUMMARY OF THE INVENTION

A first object of the present invention is to provide a viable probiotic microsphere that satisfies most of these above-mentioned needs.

The object of the present invention is also to provide enteric and moisture barrier coating compositions and processes for stabilizing and targeting probiotic microspheres into specific regions of the intestinal tract.

In accordance with the invention, this object is achieved in a viable and stable probiotic formulation for intestinal targeting, comprising a plurality of probiotic microspheres each comprising: a core comprising one or more probiotic bacteria, a cellulosic excipient, a disintegrant and one or more additives; and an enteric coating capable of being resistant to gastric fluids.

Each probiotic microsphere is characterized in that it has a residual moisture level of less than 5% and a water activity (a_{w}) between 0.1 and 0.5, and more preferably a residual moisture level of less than 2% and a water activity (a_{w}) between 0.15 and 0.35%. The Applicants have discovered that the viability of bacteria in the probiotic formulation is greatly increased by adjusting the water activity (a_{w}) and residual moisture level to specific values as described above.

A probiotic formulation in accordance with the present invention shows several advantages, namely the following: it shows no reduction in viable bacteria after 1 hour exposure to simulated gastric fluids and it shows less than 1 log loss in cfu/g at 18 months room temperature.

Preferably, the microsphere also comprises a moisture protective, and controlled disintegration non-enteric coating as an undercoat to the enteric coating.

The non-enteric coating preferably comprises one or more non-enteric agents selected from the group consisting of polyvinyl alcohol (PVA), hydroxypropyl methylcellulose (HPMC), hydroxypropyl cellulose (HPC), sodium carboxymethyl cellulose (Na-CMC), ethylcellulose (EC), waxes, fatty acids, fatty alcohols, fatty esters; and a plasticizing agent selected from the group consisting of diethyl phthalate, dibutyl sebacate, triethyl citrate, acetyltriethyl citrate, tributyl citrate and polyethylene glycol. Preferably, the plasticizing agent is selected from the group consisting of triethyl citrate and diethyl phthalate.

Also preferably, the cellulosic excipient of the probiotic microspheres is a microcrystalline cellulose (MCC) which has a degree of polymerization (DP) from 165 to 365 and a mean diameter from 45 to 180 μm. More preferably, the MCC has a DP from 220 to 230 and a mean diameter from 45 to 55 μm.

The enteric coating preferably comprises an enteric coating agent selected from the group consisting of polymers or copolymers of acrylic acid and/or methacrylic acid and/or their esters, cellulose acetate phthalate, polyvinyl acetate phthalate and shellac; and a plasticizing agent such as described above. More preferably, the enteric coating agent is selected from the group of methacrylic acid-ethyl acrylate copolymer and cellulose acetate phthalate.

The additives of the probiotic formulation preferably comprise one or more stabilizer and one or more disintegrant. In that case, the stabilizer is selected from the group consisting of glycerol, non-fat skim milk powder, ascorbic acid, anthocyanidins, flavanols, betaine, nicotinin acid, peptone, tryptone, cysteine, sodium chloride, trehalose, sucrose, short-chain fructo-oligosaccharides (scFOS), oligofructose, whey protein isolate, adonitol, meat extract and yeast extract, and more preferably from the group consisting of peptone, tryptone and scFOS. The disintegrant is preferably selected from the group consisting of croscarmelose sodium, crospovidone, sodium starch glycolate, alginic acid and starch and more preferably the disintegrant is croscarmelose sodium.

The probiotic formulation preferably comprises probiotic bacteria selected from the group consisting of Lactobacillus, Bifidobacterium, Enterococcus, Propionibacterium, Bacillus and Streptococcus and more preferably from the group consisting of Lactobacillus and Bifidobacterium.

In accordance with a preferred aspect of the invention, the core of the probiotic formulation comprises in weight percentage of its total dry weight: from 1 to 10% of probiotic bacteria; from 50 to 90% of MCC; from 0.1% to 30% of stabilizer; and from 0.1% to 5% of disintegrant.

In accordance with another preferred aspect of the invention, the probiotic formulation comprises in weight percentage of its total dry weight of each microsphere from 5 to 30% of said non-enteric coating; and from 5 to 30% of said enteric coating. More preferably, the probiotic formulation comprises from 10 to 20% of the non-enteric coating. Most preferably also, the probiotic formulation comprises 10 to 20% of the enteric coating.

In accordance with another preferred aspect of the invention, the core of the microsphere has a diameter ranging form 150 to 3000 μm. More preferably the diameter of the core ranges from 425 to 2000 μm.

The invention also provides for a process for preparing a probiotic formulation, which comprises the following steps: dry blending a microcrystalline cellulose (MCC) with a disintegrant; granulating the mixture of MCC and disintegrant with an aqueous dispersion comprising a lyophilized probiotic powder, stabilizers and purified water in order to form an extrudable paste; extruding the extrudable paste in the form of segments; spheronizing of the segments to form the core of microspheres; drying the cores to a residual moisture content of less than 5% and a water activity (a_{w}) between 0.1 and 0.5; and coating the microspheres and preferably to a residual moisture content of less than 2% and a water activity (a_{w}) between 0.15 and 0.35%. This process gives less than 1.5 loss of log colony-forming units (cfu) per gram on a dry basis at the end of the coating step.

In accordance with another preferred aspect of the invention, the cores obtained in the spheronizing step have a diameter ranging from 150 to 3000 μm. More preferably the diameter of the core ranges from 425 to 2000 μm.

The step of coating preferably comprises the steps of: covering the cores with a moisture protection and controlled disintegration non-enteric coating; and covering the non-enteric coating with an enteric coating capable of being resistant to gastric fluids.

Other objects and advantages of the present invention will be apparent upon reading the following non-restrictive description of several preferred embodiments made with reference to the accompanying examples.

### DETAILED DESCRIPTION OF PREFERRED MODES OF REALISATION OF THE INVENTION

Core Compositions

The core of the microspheres of the invention comprises one or more probiotic bacteria, a cellulosic excipient which is preferably a microcrystalline cellulose (MCC). It also further preferably comprises a disintegrant and one or more stabilizers selected from a group of substances known in the art for their protective action in freeze-drying or spray drying of bacteria.

Probiotics

Preferably, the probiotic bacteria are chosen from the genera Lactobacillus, Bifidobacterium, Enterococcus, Propionibacterium, Bacillus and Streptoccocus. More preferably, the probiotic bacteria are chosen from Lactobacillus and Bifidobacterium. The invention is not, however, limited to these particular microorganisms. The person skilled in the art would understand and recognize those microorganisms that may be included in the core compositions of the invention. Probiotic bacteria, as lyophilized preparations in powder form, are readily available commercially.

Microcrystalline Cellulose

Preferably, the microcrystalline cellulose (MCC) is present in the core composition at a concentration of about 50 to about 90%. Preferably, the microcrystalline cellulose is chosen from a group with a degree of polymerization (DP values) in the range 166 to 365 and mean diameters from 50 to 180 μm. More preferably, the microcrystalline cellulose is chosen from a group with DP in the range of 200 to 250 and mean diameters from 50 to 90 μm, and even more preferably from a group with DP in the range of 220 to 230 and mean diameters from 45 to 55 μm. A person skilled in the art could identify suppliers of these ingredients.

Stabilizers

Preferably the stabilizers are present in the core composition at a concentration of about 0.1% to about 30%. Preferably, the stabilizers are chosen from glycerol, non-fat skim milk powder, ascorbic acid, nicotinic acid, anthocyanidins, flavanols, betaine, peptone, tryptone, cysteine, sodium chloride, trehalose, sucrose, short-chain fructo-oligosaccharides (scFOS), oligofructose, whey protein isolate, adonitol, meat extract and yeast extract. More preferably, the stabilizers are chosen from peptone, tryptone and scFOS. Oligosaccharides serve an additional function as selective nutrients for resident lactic acid bacteria. These stabilizers are available commercially from various suppliers and most are used as protective solutes in the lyophilization of bacteria. A person skilled in the art would readily identify the suppliers of these ingredients.

Disintegrant

Preferably the disintegrant is present in the core composition at a concentration of about 0.1% to about 5%. Preferably the disintegrant is chosen from croscarmelose sodium, crospovidone, sodium starch glycolate, alginic acid, starch and other disintegrants known to those skilled in the art. More preferably, the disintegrant is croscarmelose sodium.

Accordingly, the probiotic microsphere core composition of the present invention preferably comprises:
a) from about 1 to about 10 percent of a lyophilized probiotic powder:
b) from about 50 to about 90 percent of microcrystalline cellulose:
c) from about 0.1 to about 30 percent of an acceptable stabilizer; and
d) from about 0.1% to about 5 percent of an acceptable disintegrant.
   Processes for Preparing Core Compositions

According to another aspect of the invention there is provided processes for entrapping and stabilizing probiotic bacteria within microcrystalline cellulose (MCC) microspheres or granules with less than 1 log loss of colony-forming units per gram at the end of the process operations. Preferably, the processes comprise the non-continuous steps of blending, granulation, extrusion, spheronization and drying, but to one skilled in the art, it is possible to conduct the entire process in a continuous fashion, for example in fluidized bed systems.

The entire processing operation for the core composition may be conducted in a facility equipped with HEPA (High Efficiency Particulate Arresting) filtration system operating at a minimum 99.97 percent efficiency.

All processing vessels, equipment and their accessories in direct contact with composition ingredients, are preferably cleaned and sterilized prior to commencing operations. Additionally, aseptic conditions are maintained throughout the processing operations in order to avoid airborne contamination.

The probiotic microsphere core compositions produced preferably have diameters within the range 100 to 3,000 82 m. Preferably, the probiotic microsphere core compositions are selected with the majority having diameters within the range between 425 to 2000 μm. More preferably, the microsphere compositions are chosen with the majority having diameters within the range 1,180 and 2,000 μm.

According to an additional preferred aspect of the invention, there is provided processes for producing microsphere core compositions having final moisture levels (Cenco Balance) less than 5 percent and water activity values ranging between 0.10 and 0.50 (Rotronic Hygrolab). More preferably, there is provided processes for producing microsphere core compositions having final moisture levels less than 2 percent and water activity values ranging between 0.15 and 0.35.

Dry Blending and Granulation Phases

Preferably, the dry blending and granulation phases are conducted in a twin-shell blender, double-cone blender, ribbon blender, planetary mixer, sigma-blade mixer or fluidized-bed granulator. More preferably, the dry blending and granulation phases are performed in a planetary mixer. Preferably, batch sizes for the dry blending and granulation phases of the operation for this disclosure are in the range of about 1,500 to 2,500 grams, more preferably in the range of about 1,750 to about 2,250 grams.

Preferably, MCC and disintegrant are pasteurized at 80° C. for 35 minutes, blended and then granulated with an aqueous dispersion comprising lyophilized probiotic powder, stabilizers and sterile distilled water under continuous mixing. Preferably the moisture content of the resulting dough or paste is in the range of about 58% to about 62% by weight, more preferably from about 59 to 60%.

Extrusion Phase

Preferably, the extrusion phase is conducted using a single-screw extruder, twin-screw extruder, ram extruder or oscillating granulator. More preferably, the extrusion phase is performed in an oscillating granulator. Preferably, the paste from the granulating phase is extruded in the form of jagged segments or strands through sieves with openings preferably in the range of from about 0.63 mm to about 3.15 mm in diameter, more preferably from about 1.6 mm to about 2.0 mm.

Spheronization and Drying Phases

Preferably, the spheronization phase is performed using a marumerizer or coating pan. More preferably, the spheronization phase for the jagged strands from the extrusion phase is conducted in a round coating pan equipped with air blower, variable heat control, variable speed and adjustable angle. Preferably, the pan operating speed is 30 RPM and hot air drying temperature is preferably in the range of about 40 to 70° C., more preferably in the range of about 50° C. to about 60° C. Preferably, the resulting spheres are dried to a residual moisture level of less than 2 percent using a combination of pan, fluidized bed and vacuum drying in order to minimize loss of viability due to lengthy heat exposure.

Coating Compositions

The coating compositions of the microspheres comprises one or more coating agents and plasticizers selected from a group of substances known in the art for their ability to provide, as a primary layer (also called non-enteric coating) a film with combined controlled disintegration and protection from moisture, and as a secondary layer (also called enteric coating), a film possessing enteric properties, i.e., resistance to gastric acid attack.

According to another aspect of the invention there is provided coating processes for probiotic microsphere compositions with less than 1.5 loss of log colony-forming units per gram on a dry basis at the end of the combined core and coating process operations.

Primary Films (Non-Enteric Coating)

Preferably the controlled disintegration and moisture protective coating agents are present in the composition at a combined concentration from about 5 to about 30 percent. More preferably, the combined films are present in the composition from about 10 to 20 percent. Preferably, the agents are chosen from aqueous dispersions of polyvinyl alcohol (PVA), hydroxypropyl methylcellulose (HPMC), hydroxypropyl cellulose (HPC), carboxymethyl cellulose (CMC) and ethylcellulose (EC). Other useful moisture protective coating agents are waxes, fatty acids, fatty alcohols, fatty esters, or the like. Any one skilled in the art could readily identify suppliers of these coating polymers.

Enteric Coating Agents

Preferably the enteric coating agents are present in the composition at a concentration from about 5 to about 30 percent. More preferably, the polymers are present in the composition from about 10 to about 20 percent. Preferably, the agents are chosen from aqueous dispersions of polymers or copolymers of acrylic acid and/or methacrylic acid and/or their esters, cellulose acetate phthalate, polyvinyl acetate phthalate, shellac, or the like. Any one skilled in the art could identify the suppliers of these coating polymers.

Plasticizing Agents

Preferably the plasticizing agents are present in the composition at a concentration from about 10 to about 35 percent of the polymer concentration used for the coating application. A person skilled in the art would know the best plasticizer-polymer combination to be used for a particular application. Preferably, the agents are chosen from diethyl phthalate, dibutyl sebacate, triethyl citrate, acetyltriethyl citrate, tributyl citrate, acetylated monoglycerides, glyceryl triacetate, or the like. More preferably, the plasticizing agents are selected from triethyl citrate and diethyl phthalate.

According to a preferred aspect of the invention, the coating compositions may be prepared and applied to the probiotic microsphere core compositions of the invention by any conventional methods known in the art. Preferably, the coating compositions are applied by a fluidized bed system with Wurster insert

The probiotic microsphere compositions obtained from the process of the invention show less than 1.5 loss of log colony-forming units per gram (cfu/g) on a dry basis at the end of the entire operations with final moisture levels less than 5 percent and water activities (a_{w}) ranging between 0.1 and 0.5. More preferably, there is provided coating processes for producing probiotic microsphere compositions with less than 1.5 log cfu/g at the end of the entire operations with final moisture levels less than 2 percent and water activities (a^{w}) ranging between 0.15 and 0.35.

The coated microsphere compositions obtained from the process demonstrate no loss in log cfu/g after 1-hour exposure to simulated gastric fluids.

The barrier protective properties of the coated probiotic microsphere compositions were estimated using 40° C. and 56% relative humidity conditions. The coated probiotic microsphere compositions demonstrated ≦1.0 log cfu/g after 4-day exposure.

The coated probiotic microspheres show a predicted (in accordance with Arrhenius relationship) loss of less than 1 log loss in cfu/g at 18 months room temperature storage. Preferably, the predicted loss is not less than 10,000,000 or log 7.0 cfu (colony-forming units) per capsule containing coated probiotic microsphere compositions after 18 months room temperature storage.

### EXAMPLES

The following examples are illustrative of the wide range of applicability of the present invention and are not intended to limit its scope. Modifications and variations can be made therein without departing from the spirit and scope of the invention. Although any method and material similar or equivalent to those described herein can be used in the practice for testing of the present invention, the preferred methods and materials are described.

Examples 1 and 2 represent microsphere cores, ranging in diameter between 425 and 1180 μm that are coated with Eudragit L30 D-55 for the purpose of Accelerated Shelf Life Testing (ASLT). Both Lactobacillus acidophilus and Bifidobacterium longum, aerobic and anaerobic bacteria respectively, are utilized as examples in this disclosure. Such formulations are ideally suited for targeting lactobacilli species to the distal regions of the small intestine.

### Example 1

### L. acidophilus Coated Microsphere Composition

The following core composition utilizes ˜4.5% short-chain fructo-oligosaccharides, peptone and tryptone as stabilizing agents and croscarmelose sodium as disintegrant. This core formulation provided microspheres with the majority within the 425 to 1180 μm diameter range. Eudragit L30 D55 and plasticizer triethyl citrate served as the enteric coating composition.

| | Weight |
|---|---|
| Ingredients | (%) |
| CORE | |
| *Microcrystalline cellulose | 76.12 |
| **Croscarmelose sodium | 0.80 |
| ***Short-chain fructo-oligosaccharides | 3.85 |
| \hat{ }Lactobacillus acidophilus (1.5 × 10¹¹ cfu/g) | 2.30 |
| \hat{ }\hat{ }Bacto ™ Peptone | 0.12 |
| \hat{ }\hat{ }Bacto ™ Tryptone | 1.92 |
| COATING | |
| \hat{ }\hat{ }\hat{ }Methacrylic acid copolymer | 12.95 |
| \hat{ }\hat{ }\hat{ }\hat{ }Triethyl citrate | 1.94 |
| TOTAL | 100.00 |

*Tabulose 101 and
**Solutab, Blanver, Sao Paulo, Brazil;
***NutraFlora scFOS; GTC Nutrition, Golden, CO;
\hat{ }Institut Rosell, Montreal, QC;
\hat{ }\hat{ }Becton Dickinson, Sparks, MD;
\hat{ }\hat{ }\hat{ }Roehm Gmbh & Co., Germany:
\hat{ }\hat{ }\hat{ }\hat{ }Morflex, Greensboro, NC.

The viability results for this lot, after various processing steps, appear in the following table and demonstrate an overall log cfu/g reduction of 0.93 after blending, granulation, extrusion, spheronization, drying and coating processes. The residual moisture content at the end of the processing operations was 1.0 percent and water activity value 0.236.

Testing of the coated microspheres in simulated gastric fluids revealed no log reduction in viable bacteria numbers after 1-hour exposure to simulated gastric fluids (see Example 6).

Viability (dried basis)

| Operation | Log cfu/gram | Log loss |
|---|---|---|
| Start | 9.53 | — |
| Blending/Granulation | 9.09 | 0.44 |
| Extrusion | 9.03 | 0.47 |
| Spheronization | 9.00 | 0.53 |
| Drying | 8.60 | 0.93 |
| Coating | 8.60 | 0.93 |

Accelerated shelf life testing of these coated microspheres was performed according to the method in Example 8. According to the Arrhenius relationship, predicted loss of viability, expressed in log cfu/g, after 18 months storage at 22° C. was 0.82, with a final predicted product concentration of log 7.78 cfu per gram.

### Example 2

### B. longum Coated Microsphere Composition

The following example is similar to Example 1 except B. longum replaces L. acidophilus.

| Ingredients | Weight (%) |
|---|---|
| CORE | |
| Microcrystalline cellulose | 80.06 |
| Croscarmelose sodium | 0.84 |
| Short-chain fructo-oligosaccharides | 4.05 |
| *Bifidobacterium longum (5 × 10¹⁰ cfu/g) | 2.42 |
| Bacto ™ Peptone | 0.12 |
| Bacto ™ Tryptone | 2.02 |
| COATING | |
| Methacrylic acid copolymer | 9.12 |
| Triethyl citrate | 1.37 |
| TOTAL | 100.00 |

*Institut Rosell, Montreal, QC

The viability results for this lot after various processing steps appear in the following table and demonstrates an overall log cfu/g reduction of 0.80 after blending, granulation, extrusion, spheronization, drying and coating processes. The residual moisture content at the end of the processing operations was 1.5 percent and water activity value 0.239.

Testing of the coated microspheres in simulated gastric fluids revealed no log reduction in viable bacteria numbers after 1-hour exposure to simulated gastric fluids (see Example 6)

Viability (dried basis)

| Operation | Log cfu/gram | Log loss |
|---|---|---|
| Start | 8.92 | — |
| Blending/Granulation | 8.73 | 0.19 |
| Extrusion | 8.67 | 0.25 |
| Spheronization | 8.55 | 0.37 |
| Drying | 8.26 | 0.66 |
| Coating | 8.12 | 0.80 |

Accelerated shelf life testing of these coated microspheres was performed according to the method in Example 8. From the Arrhenius relationship, predicted loss of viability, expressed in log cfu/g, after 18 months storage at 22° C. was 0.52, with a final predicted product concentration at this time period of log 7.60 cfu per gram.

Examples 3 and 4 demonstrate coated probiotic microsphere compositions that are designed to withstand 1 hour exposure to simulated gastric fluid testing and to lose less than 1 log cfu/g viability after 4 days exposure to 40° C. and 56% relative humidity conditions. Only Bifidobacterium longum is utilized as examples in this disclosure. Such compositions are useful for enhancing viability, stability and targeting bifidobacteria species to the colonic region.

### Example 3

### B. longum Coated Microsphere Composition

The following core composition utilizes ˜23.5% short-chain fructo-oligosaccharides, peptone and tryptone as stabilizing agents and croscarmelose sodium as disintegrant. This core formulation provided microspheres with the majority falling within the 1180 to 2000 μm diameter range. Opadry AMB serves as the primary barrier coat and Eudragit L30 D55 as secondary enteric coat, respectively.

| Ingredients | Weight (%) |
|---|---|
| CORE | |
| Microcrystalline cellulose | 52.25 |
| Croscarmelose sodium | 0.75 |
| Short-chain fructo-oligosaccharides | 16.76 |
| Bifidobacterium longum (5 × 10¹⁰ cfu/g) | 1.58 |
| Bacto ™ Peptone | 0.09 |
| Bacto ™ Tryptone | 1.32 |
| COATING | |
| *Opadry AMB | 10.00 |
| Methacrylic acid copolymer | 15.00 |
| Triethyl citrate | 2.25 |
| TOTAL | 100.00 |

*Colorcon, West Point, PA

The viability results for this lot after various processing steps appear in the following table and demonstrate an overall log cfu/g reduction of 1.27 after blending, granulation, extrusion, spheronization, drying and coating processes. The residual moisture content at the end of the processing operations was 1.5 percent.

Testing of the coated microspheres in simulated gastric fluids revealed no log reduction in viable bacteria numbers after 1-hour exposure to simulated gastric fluids (see Example 6). The coated microspheres also passed the barrier limit test (Example 7).

Viability (dried basis)

| Operation | Log cfu/gram | Cum. Log loss |
|---|---|---|
| Start | 8.83 | — |
| Blending/Granulation | 8.47 | 0.36 |
| Extrusion | 8.39 | 0.44 |
| Spheronization | 8.22 | 0.61 |
| Drying | 8.07 | 0.76 |
| Coating | 7.56 | 1.27 |

### Example 4

### B. longum Coated Microsphere Composition

The following example uses the same microsphere cores from Example 3 but Sepifilm LP 007 replaces Opadry AMB as a primary layer film and Aquacoat CPD 30 replaces Eudragit L 30 D-55 as enteric film. Diethyl phthalate replaces triethyl citrate as plasticizer.

| Ingredients | Weight (%) |
|---|---|
| CORE | |
| Microcrystalline cellulose | 51.28 |
| Croscarmelose sodium | 0.74 |
| Short-chain fructo-oligosaccharides | 16.45 |
| Bifidobacterium longum (5 × 10¹⁰) | 1.55 |
| Bacto ™ Peptone | 0.09 |
| Bacto ™ Tryptone | 1.29 |
| COATING | |
| *Sepifilm LP 007 | 10.00 |
| **Aquacoat CPD 30 | 15.00 |
| ***Diethyl phthalate | 3.60 |
| TOTAL | 100.00 |

*Seppic Inc., Fairfield, NJ;
**Colorcon, West Point, PA;
***A&C Chemicals, Montreal, QC.

The viability results for this lot after the coating operations, including the processing steps for microsphere cores from Example 2, demonstrate an overall log reduction of 1.22. The residual moisture content at the end of the coating operations was 0.80 percent.

Testing of the coated microspheres in simulated gastric fluids revealed no log reduction in viable bacteria numbers after 1-hour exposure to simulated gastric fluids. The coated microspheres also passed the barrier limit test (Example 7).

### Example 5

### Microbiology Assay Methods

Lactic acid bacteria viability determinations are conducted by collecting appropriate samples in sterile whirl pack bags. Each sample (1.0 gram) is put into a stomacher bag along with 0.1% peptone solution (99.0 mL) to form the first dilution (1:100). The bag is then placed in the Stomacher and the latter permitted to operate at 230 RPM for 10 minutes to assure adequate homogenization of the bacteria suspension.

Decimal serial dilutions and 0.1 mL aliquots of each dilution are inoculated on Reinforced Clostridial media (RCM). All inoculations are performed in duplicate. RCM plates are incubated at 37° C. for 48 hours in an anaerobic jar for Bifidobacterium longum and for 72 to 96 hours in an aerobic environment for Lactobacillus acidophilus. Colony-forming units are counted in the plates containing between 30 and 300 colonies and calculations made to determine viable cells concentration in the original sample. Values are adjusted according to moisture contribution to obtain bacteria concentration on a dry weight basis. Viability loss represents the difference between the initial and final bacteria concentration in the mix.

### Example 6

### Enteric Testing Method

The test procedure uses a slightly-modified in vitro assembly and operating conditions utilized in predicting bioavailability of drug dosage forms (Simmons et al, Canadian Journal of Pharmaceutical Sciences, 1980, 15(2), 26-29). The modification was in the design of the disintegration chamber where the apertures were reduced in size in order to prevent the microspheres from being expelled into the surrounding medium. Hydrodynamic conditions are much more turbulent in the disintegration chamber (e.g., stomach) than in the surrounding medium. The test medium is simulated gastric fluid as described in USP XXV and maintained at 37° C.

One cellulose capsule (#0), containing ˜450 mg microspheres, is placed in the disintegrating chamber and the assembly immersed into the simulated gastric juice. Following capsule disintegration (˜3 minutes), the released microspheres are exposed to the turbulent conditions for 1 hour, isolated, dispersed in phosphate buffer ˜pH 7.3 to dissolve the enteric coating and enumerated for viability retention.

### Example 7

### Barrier Test

A desiccator containing a saturated solution of magnesium nitrate (Mg(NO₃)₂.6H₂O) is incubated at 40° C. and allowed to equilibrate until relative humidity (RH) reached 56 percent. A plastic weighing boat containing four microsphere-containing cellulose capsules (#0, ˜450 mg microspheres per capsule) is than placed in the desiccator. After a period of 4 days, the capsules are removed and the microspheres enumerated for viability retention as in Example 6. Log cfu/g losses should not exceed 1.0 after 4 days exposure.

### Example 8

### Accelerated Shelf Life Testing (ASLT)

Accelerated shelf life testing of coated microspheres was performed according to a modified method of Mitic et al (Cryobiology, 1974, 11: 116-120). The coated microspheres were filled into sixteen #0 cellulose (HPMC) capsules, and then packaged along with cotton plugs in 40 mL HDPE bottles with polypropylene caps. The bottles were than placed in incubators maintained at 50, 60 and 70° C., and at predetermined hourly intervals, samples removed for viability testing.

While several embodiments of the invention have been demonstrated, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention as described herein.

1. A viable and stable probiotic formulation for intestinal targeting, comprising:
a plurality of probiotic microspheres each comprising:
a core comprising one or more probiotic bacteria, a cellulosic excipient, a disintegrant and one or more additives; and
an enteric coating capable of being resistant to gastric fluids, having a residual moisture level of less than 5% and a water activity (a_{w}) between 0.1 and 0.5.

2. The probiotic formulation according to claim 1, wherein said residual moisture level is less than 2%.

3. The probiotic formulation according to claim 1, wherein said water activity (a_{w}) is between 0.15 and 0.35.

4. The probiotic formulation according to claim 1, having no reduction in viable bacteria after 1 hour exposure to simulated gastric fluids.

5. The probiotic formulation according to claim 4, comprising:
a moisture protective and controlled disintegration non-enteric coating as an undercoat to the enteric coating.

6. The probiotic formulation according to claim 5, wherein said non-enteric coating comprises:
one or more non-enteric agents selected from the group consisting of polyvinyl alcohol (PVA), hydroxypropyl methylcellulose (HPMC), hydroxypropyl cellulose (HPC), sodium carboxymethyl cellulose (Na—CMC), ethylcellulose (EC), waxes, fatty acids, fatty alcohols, fatty esters; and
a plasticizing agent selected from the group consisting of diethyl phthalate, dibutyl sebacate, triethyl citrate, acetyltriethyl citrate, tributyl citrate and polyethylene glycol.

7. The probiotic formulation according to claim 6, wherein said plasticizing agent is selected from the group consisting of triethyl citrate and diethyl phthalate.

8. The probiotic formulation according to claim 6, wherein the cellulosic excipient is a microcrystalline cellulose (MCC).

9. The probiotic formulation according to claim 8, wherein said MCC has a degree of polymerization (DP) from 165 to 365 and a mean diameter from 45 to 180 μm.

10. The probiotic formulation according to claim 9, wherein said DP is from 220 to 230 and said mean diameter is from 45 to 55 μm.

11. The probiotic formulation according to claim 1, wherein said enteric coating comprises:
an agent selected from the group consisting of polymers or copolymers of acrylic acid and/or methacrylic acid and/or their esters, cellulose acetate phthalate, polyvinyl acetate phthalate and shellac; and
a plasticizing agent being selected from the group consisting of diethyl phthalate, dibutyl sebacate, triethyl citrate, acetyltriethyl citrate and tributyl citrate.

12. The probiotic formulation according to claim 11, wherein the enteric coating agent is selected from the group of methacrylic acid-ethyl acrylate copolymer and cellulose acetate phthalate.

13. The probiotic formulation according to claim 11, wherein said plasticizing agent is selected from the group consisting of triethyl citrate and diethyl phthalate.

14. The probiotic formulation according to claim 8, wherein said additives comprise one or more stabilizer.

15. The probiotic formulation according to claim 14, wherein the core comprises in weight percentage of the total dry weight of the core:
from 1 to 10% of said probiotic bacteria;
50 to 90% of said MCC;
from 0.1% to 30% of said stabilizer; and
from 0.1% to 5% of said disintegrant.

16. The probiotic formulation according to claim 15, wherein it comprises in weight percentage of the total dry weight of each microsphere:
from 5 to 30% of said non-enteric coating; and
from 5 to 30% of said enteric coating.

17. The probiotic formulation according to claim 16, wherein the non-enteric coating is present at a concentration from 10 to 20%.

18. The probiotic formulation according to claim 16, wherein the enteric coating is present at a concentration from 10 to 20%.

19. The probiotic formulation according to claim 1, wherein said core of the microspheres has a diameter ranging from 150 to 3000 μm.

20. The probiotic formulation according to claim 19, wherein said core has a diameter ranging from 425 to 2000 μm.

21. The probiotic formulation according to claim 1, wherein the probiotic bacteria are selected from the group consisting of Lactobacillus, Bifidobacterium, Enterococcus, Propionibacterium, Bacillus and Streptococcus.

22. The probiotic formulation according to claim 21, wherein the probiotic bacteria are selected from the group consisting of Lactobacillus and Bifidobacterium.

23. The probiotic formulation according to claim 15, wherein said stabilizer is selected from the group consisting of glycerol, non-fat skim milk powder, ascorbic acid, anthocyanidins, flavanols, betaine, nicotinin acid, peptone, tryptone, cysteine, sodium chloride, trehalose, sucrose, short-chain fructo-oligosaccharides (scFOS), oligofructose, whey protein isolate, adonitol, meat extract and yeast extract.

24. The probiotic formulation according to claim 23, wherein the stabilizer is selected from the group consisting of peptone, tryptone and scFOS.

25. The probiotic formulation according to claim 15, wherein the disintegrant is selected from the group consisting of croscarmelose sodium, crospovidone, sodium starch glycolate, alginic acid and starch.

26. The probiotic formulation according to claim 25, wherein said disintegrant is croscarmelose sodium.

27. A process for preparing a probiotic formulation as defined in claim 1, said process comprising the steps:
dry blending a microcrystalline cellulose (MCC) with a disintegrant;
granulating said mixture of MCC and disintegrant with an aqueous dispersion comprising a lyophilized probiotic powder, stabilizers and purified water in order to form an extrudable paste;
extruding said extrudable paste in the form of segments;
spheronizing segments to form cores as defined in claim 1;
drying the cores to a residual moisture level of less than 5% and a water activity (a_{w}) between 0.1 and 0.5; and
coating said cores to obtain the microspheres;
said process giving less than 1.5 loss of log colony-forming units (cfu) per gram on a dry basis at the end of the coating steps.

28. The process according to claim 27, wherein said residual moisture level is less than 2%.

29. The process according to claim 27, wherein said water activity (a_{w}) is between 0.15 and 0.35.

30. The process according to claim 27, wherein said MCC has a degree of polymerization (DP) from 165 to 365 and a mean diameter from 45 to 180 μm.

31. The process according to claim 30, wherein said MCC has a degree of polymerization (DP) from 220 to 230 and a mean diameter from 45 to 55 μm.

32. The process according to claim 27, wherein said disintegrant is selected from the group consisting of croscarmelose sodium, crospovidone, sodium starch glycolate, alginic acid and starch.

33. The process according to claim 27, wherein said probiotic powder is selected from the group consisting of Lactobacillus, Bifidobacterium, Enterococcus, Propionibacterium, Bacillus and Streptococcus.

34. The process according to claim 33, wherein said probiotic bacteria are selected from the group consisting of Lactobacillus and Bifidobacterium.

35. The process according to claim 27, wherein the cores obtained in the spheronizing step have a diameter ranging from 150 to 3000 μm.

36. The process according to claim 35, wherein said diameter of the cores ranges from 425 to 2000 μm.

37. The process according to claim 27, wherein the step of coating comprises the steps of:
covering said cores with a moisture protection and controlled disintegration non-enteric coating; and
covering said non-enteric coating with an enteric coating capable of being resistant to gastric fluids.

38. The process according to claim 37, wherein said non-enteric coating comprises:
one or more non-enteric agents selected from the group consisting of polyvinyl alcohol (PVA), hydroxypropyl methylcellulose (HPMC), hydroxypropyl cellulose (HPC), sodium carboxymethyl cellulose (Na—CMC), ethylcellulose (EC), waxes, fatty acids, fatty alcohols, fatty esters; and
a plasticizing agent selected from the group consisting of diethyl phthalate, dibutyl sebacate, triethyl citrate, acetyltriethyl citrate, tributyl citrate and polyethylene glycol.

39. The process according to claim 37, wherein said enteric coating comprises:
an agent selected from the group consisting of polymers or copolymers of acrylic acid and/or methacrylic acid and/or their esters, cellulose acetate phthalate, polyvinyl acetate phthalate and shellac; and
a plasticizing agent being selected from the group consisting of diethyl phthalate, dibutyl sebacate, triethyl citrate, acetyltriethyl citrate and tributyl citrate.
